(19) United States
(12) Patent Application Publication
(10) Pub. No.: US 2023/0364081 A1
(43) Pub. Date: Nov. 16, 2023

(54) METHOD AND FORMULATION FOR TREATING MALE PREMATURE EJACULATION

(71) Applicants: Taghogho Agarin, San Jose, CA (US); Michael Callis, Santa Maria, CA (US); Robert McLay, Chula Vista, CA (US)

(72) Inventors: Taghogho Agarin, San Jose, CA (US); Michael Callis, Santa Maria, CA (US); Robert McLay, Chula Vista, CA (US)

(73) Assignee: Zyvran Pharmaceuticals Inc., Los Angeles, CA (US)

(21) Appl. No.: 18/195,416

(22) Filed: May 10, 2023

Related U.S. Application Data
(60) Provisional application No. 63/340,357, filed on May 10, 2022.

(51) Int. Cl. A61K 31/4985; A61K 31/404; A61K 31/4525

(52) U.S. Cl. CPC A61K 31/4985; A61K 31/404; A61K 31/4525

(57) ABSTRACT

A triple drug low dose combination therapy formulation for treating male premature ejaculation may include a phosphodiesterase 5 inhibitor (PDE5i), such as Tadalafil; an alpha-1 adrenoreceptor (alpha-1a) selective antagonist, such as Silodosin; and a selective serotonin reuptake inhibitor (SSRI), such as Paroxetine. A method of treating premature ejaculation in men may include administering a therapeutically effective dosage of the formulation to a patient in need thereof.

### RELATED APPLICATION

This application claims priority to provisional patent application U.S. Ser. No. 63/340,357 filed on May 10, 2022, the entire contents of which is herein incorporated by reference.

### BACKGROUND

The embodiments described herein relate generally to therapies and treatments for male premature ejaculation and, more particularly, to a triple drug, low dose combination therapy for the treatment of male premature ejaculation.

Premature ejaculation (PE) is one of the most common forms of male sexual dysfunction. Some studies suggest that between about 20-25% of males have complained about PE at some point in their lives. PE is commonly defined as ejaculation before, shortly after, or upon penetration, and before the man or his partner wishes. The most common clinical definition of PE is ejaculation within 1 minute of vaginal penetration. PE may be lifelong, starting with first sexual experience (primary) or acquired later (secondary). Some males who worry about getting and maintaining an erection will overstimulate, making PE more likely.

Various regimens have been proposed and used for the treatment of PE. The existing treatments include behavioral, topical, and systemic medications. Numbing creams can reduce sensation and, thereby, slow down orgasm, but they are far from ideal if the individual worries about getting and maintaining an erection. Behavioral techniques can be helpful by teaching the individual to slow down and not finish as quickly. However, its success is often intermittent or not at all. If overstimulation from fears of losing one's erection is primarily the reason for PE, then erection medications and other “off-label” uses of other medications may be helpful.

The effectiveness of PE treatment is generally assessed by measuring time from penetration to ejaculation. Other measures of effectiveness include improved sexual satisfaction, subjective perceived control over timing of ejaculation, relationship satisfaction, self-esteem, and quality of life. As with any medical intervention, the positive benefits are balanced against side effects and safety concerns.

Some medical interventions demonstrate some improvement in extending the time after penetration, intra-vaginal ejaculatory latency time (IELT). These include topical anesthetics, selective serotonin reuptake inhibitors (SSRIs), serotonin-noradrenaline reuptake inhibitors (SNRIs), tricyclic antidepressants, phosphodiesterase-5 inhibitors (PED5i), opioid-based analgesics (e.g., tramadol), alpha-blockers (e.g., selective alpha₁-adrenoceptor antagonists), combinations with a drug from two or more of the above classes, acupuncture, and behavioral therapies—including stop-start, the squeeze technique, and yoga, each of which has had some success. There is also an FDA approved medical device to help with the start-stop behavioral technique.

Issues with the conventional treatment methods, formulations, and device are various. For example, to achieve delay with one drug class, the drug needs to be given at higher doses, which often results in intolerable side effects. These side effects often cause patients to stop taking the drug.

Therefore, what is needed is a method and formulation for providing superior and more extended delay in male ejaculation while reducing or preventing intolerable side effects.

### SUMMARY

Some embodiments of the present disclosure include a triple drug low dose combination therapy formulation for treating male premature ejaculation. The formulation may include a phosphodiesterase 5 inhibitor (PDE5i), such as Tadalafil; an alpha-1 adrenoreceptor (alpha-1a) selective antagonist, such as Silodosin; and a selective serotonin reuptake inhibitor (SSRI), such as Paroxetine. A method of treating premature ejaculation in men may include administering a therapeutically effective dosage of the formulation to a patient in need thereof.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The formulation of the present disclosure may be used as a low dose combination therapy for treatment of male premature ejaculation and may comprise the following elements. This list of possible constituent elements is intended to be exemplary only, and it is not intended that this list be used to limit the formulation of the present application to just these elements. Persons having ordinary skill in the art relevant to the present disclosure may understand there to be equivalent elements that may be substituted within the present disclosure without changing the essential function or operation of the formulation.

The various elements of the present disclosure may be related in the following exemplary fashion. It is not intended to limit the scope or nature of the relationships between the various elements, and the following examples are presented as illustrative examples only.

By way of example, some embodiments of the present disclosure include a method and pharmaceutical formulation for the treatment of male premature ejaculation, wherein the formulation comprises a triple drug low dose combination.

More specifically, the pharmaceutical formulation of the present disclosure comprises a phosphodiesterase 5 inhibitor (PDE5i), an alpha-blocker, and a selective serotonin reuptake inhibitor (SSRI) combined in a single dosage form. The drug agents include the non-salt form of the drug, either of the stereoisomers of a compound comprising a mirror of the other enantiomers of the drug, and salts of the drug.

In embodiments, the PDE5i may comprise Tadalafil, the alpha-blocker may comprise an alpa-1 adrenoreceptor (alpha-1a) selective antagonist, such as silodosin, and the SSRI may comprise Paroxetine. The combined dosages may vary in amount and administration. For example, in some embodiments, the formulation may comprise about 5 to about 20 wt. % PDE5i, about 1 to about 4 wt. % alpha-blocker, and about 10 to about 12.5 wt. % SSRI.

Tadalafil delays ejaculation on its own and prolongs intra-vaginal ejaculatory latency time (IELT). Higher doses are more effective but can produce unwanted side effects, such as facial flushing and myalgia.

Silodosin is superior in delaying ejaculation compared to other drugs in its class, but in one study does not prolong IELT sufficiently at a 4 mg dose when compared to established norms. In another different, but related study, it causes an ejaculation in about 50% of people taking it if the dose were increased to 8 mg. In addition, the 8 mg dosing may cause a reduction in orgasmic feeling, and retrograde ejaculation.

Paroxetine has been known to show superior efficacy for delaying ejaculations based on meta-analysis of randomized studies compared to placebo, fluoxetine, and Lexapro. However, it is less tolerable compared to other medications in its class, especially at higher doses.

The combination of Tadalafil, Silodosin, and Paroxetine in a single pill formulation may allow for the most delay in ejaculation using relatively small doses of each, thus preventing the unwanted side effects obtainable when a single drug is given at a high dose.

A method of treating premature ejaculation in men by producing a more extended IELT while minimizing side effects may comprise administering a therapeutically effective dosage of a formulation to a patient in need, wherein the formulation comprises Tadalafil, Silodosin, and Paroxetine. The formulation may be provided once or twice daily in very low doses of the tripe combination. Alternatively, the formulation may be provided on-demand to increase ejaculatory latency time.

In a particular embodiment, a therapeutically effective dosage of the formulation of the present disclosure may comprise about 0.5 to 8 mg Silodosin, about 2 to 40 mg Tadalafil, and about 2 to 60 mg Paroxetine. As mentioned above, the formulation may be combined into a single dosage pill, which may be administered on-demand or in a daily formulation. Alternatively, the formulation may be administered in tablets, capsules, oral disintegrating tablets, oral disintegrating strips, oral dispersible films, liquid solutions, extended release preparations, controlled release forms, lozenges, films, nasal sprays, patches or topical cream applications.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

What is claimed is:

1. A triple drug low dose combination therapy formulation for treating male premature ejaculation, the formulation comprising:
a phosphodiesterase 5 inhibitor (PDE5i);
an alpha-blocker; and
a selective serotonin reuptake inhibitor (SSRI).

2. The formulation of claim 1, wherein the alpha-blocker is an alpha-1 adrenoreceptor (alpha-1a) selective antagonist.

3. The formulation of claim 2, wherein:
the PDE5i comprises Tadalafil;
the alpha-1a selective antagonist is Silodosin; and
the SSRI is Paroxetine.

4. The formulation of claim 1, wherein the formulation comprises:
about 5 to about 20 wt. % PDE5i;
about 1 to about 4 wt. % alpha-blocker; and
about 10 to about 12.5 wt. % SSRI.

5. The formulation of claim 1, wherein the formulation is formed into a member selected from the group consisting of a single dosage pill and a combination of the component pills packaged together.

6. A method of treating premature ejaculation in men, the method comprising:
administering a therapeutically effective dosage of a formulation to a patient in need, wherein:
the formulation comprises:
a phosphodiesterase 5 inhibitor (PDE5i);
an alpha-blocker; and
a selective serotonin reuptake inhibitor (SSRI).

7. The method of claim 6, wherein:
the PDE5i is Tadalafil;
the alpha-blocker is Silodosin; and
the SSRI is Paroxetine.

8. The method of claim 6, wherein a therapeutically effective dosage of the formulation comprises:
about 0.5 to about 8 mg PDE5i;
about 2 to about 40 mg alpha-blocker; and
about 2 to about 60 mg SSRI.

9. The method of claim 6, wherein the formulation is combined into a single dosage pill.
